SEQUENCE LISTING (14 sequences)

SEQ ID NO: 1 - DNA, 2799 bp, Homo sapiens
atgccctcccctctgtgtggccggaatccttgcctgtggctttctcctgggctgctggggaccctcccatttccagcagagttgtcttcaggctttggagccacaggccgtgtcttcttacttgagccctggtgctcccttaaaagaaccatcgccctctgctctccctctccccctccaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgccccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccagtgtgtaaaaaagcagattcccgggtcctctgcatattccctgaatcaggacttccctgtgttgggcctgagaaaccgcaccgtaaccaacacaggcttgcggcactggccagatgtgggcatcgagggggcagctcagccagtgtcctgtgcactgtgctatccccagagcctggtacaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctactgggcggtgggagacttcggcccatgcagcgcctcctgtgggggcggcctgcgggagcggccagtgcgctgcgtggaggcccagggcagcctcctgaagacattgcccccagcccggtgcagagcaggggcccagcagccagctgtggcgctggaaacctgcaacccccagccctgccctgccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccat

SEQ ID NO: 2 - protein, 933 aa, Homo sapiens
MPSPLCGRNPCLWLSPGLLGTLPFPAELSSGFGATGRVFLLEPWCSLKRTIALCSPSPPPGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQCVKKQIPGSSAYSLNQDFPVLGLRNRTVTNTGLRHWPDVGIEGAAQPVSCALCYPQSLVQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGH

SEQ ID NO: 3 - protein, 951 aa, Mus musculus
MGDVQRAARSRGSLSAHMLLLLLASITMLLCARGAHGRPTEEDEELVLPSLERAPGHDSTTTRLRLDAFGQQLHLKLQPDSGFLAPGFTLQTVGRSPGSEAQHLDPTGDLAHCFYSGTVNGDPGSAAALSLCEGVRGAFYLQGEEFFIQPAPGVATERLAPAVPEEESSARPQFHILRRRRRGSGGAKCGVMDDETLPTSDSRPESQNTRNQWPVRDPTPQDAGKPSGPGSIRKKRFVSSPRYVETMLVADQSMADFHGSGLKHYLLTLFSVAARFYKHPSIRNSISLVVVKILVIYEEQKGPEVTSNAALTLRNFCNWQKQHNSPSDRDPEHYDTAILFTRQDLCGSHTCDTLGMADVGTVCDPSRSCSVIEDDGLQAAFTTAHELGHVFNMPHDDAKHCASLNGVTGDSHLMASMLSSLDHSQPWSPCSAYMVTSFLDNGHGECLMDKPQNPIKLPSDLPGTLYDANRQCQFTFGEESKHCPDAASTCTTLWCTGTSGGLLVCQTKHFPWADGTSCGEGKWCVSGKCVNKTDMKHFATPVHGSWGPWGPWGDCSRTCGGGVQYTMRECDNPVPKNGGKYCEGKRVRYRSCNIEDCPDNNGKTFREEQCEAHNEFSKASFGNEPTVEWTPKYAGVSPKDRCKLTCEAKGIGYFFVLQPKVVDGTPCSPDSTSVCVQGQCVKAGCDRIIDSKKKFDKCGVCGGNGSTCKKMSGIVTSTRPGYHDIVTIPAGATNIEVKHRNQRGSRNNGSFLAIRAADGTYILNGNFTLSTLEQDLTYKGTVLRYSGSSAALERIRSFSPLKEPLTIQVLMVGHALRPKIKFTYFMKKKTESFNAIPTFSEWVIEEWGECSKTCGSGWQRRVVQCRDINGHPASECAKEVKPASTRPCADLPCPHWQVGDWSPCSKTCGKGYKKRTLKCVSHDGGVLSNESCDPLKKPKHYIDFCTLTQCS

SEQ ID NO: 4 - DNA, 2217 bp, Homo sapiens
cagcttcggcctggagcacgacggcgcgcccggcagcggctgcggccccagcggacacgtgatggcttcggaacggcgccgccccgcgccggcctcgcctggtccccctgcagccgccggcagctgctgagcctgctcagacccgtccctccgtcgccgctccctctgctggccacccacctctgcgccggcaggagccttagtcttggtcccagccaagagccggctcctggtggggggcgcgggccgagaactcctgttcccactcacaaaaggccacgcttccaaacgcttccatcctcgtgcccactcctccgtcccgcctcctcccggtgtacaccccgggactgagccgggcctgagccgggccttgtcgcagcgcatgacgggcgcgctggtgtgggacccgccgcggcctcaacccgggtccgcggggcacccgcggaatgcgcacctgggcctctactacagcgccaacgagcagtgccgcgtggccttcggccccaaggctgtcgcctgcaccttcgccagggagcacctggtgagtctgccggcggtggcctgggattggctgtgaggtccctccgcatcacccagctcacgtccccccaaacgtgcatggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgcggcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctg

SEQ ID NO: 5 - DNA, 592 bp, Homo sapiens
  misc_feature (514)..(514): n=a, c, g or t
gcggccgctacctgagcaggctcagcagctgccggcggctgcagggggaccaggcgaggccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaacctagtgatatagaggaccaggtcagcatggccaggatccgtgtcgtcctcagggttgatggtctggctccacccacagacgctcagcagggacgaggtgaggttggctgtgatatttggagcaccctcaggctctgtcagaatgaccatcttcaccaggtgcacccgaaactgagcccccagggactggtcccgaagcagttctgccccgatgttganggttgtgagcacatagcgctctgtgtcctcctggtgagcctggaagacatccgtgcccacggncaccagcagctgcat

SEQ ID NO: 6 - DNA, 562 bp, Homo sapiens
  misc_feature (361)..(361): n=a, c, g or t
gcagctgccggcggctgcagggggaccaggcgaagccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaacctagtgatatagaggaccaggtcagcatggccaggatccgtgtcgtcctcagggttgatggtctggctccacccacagacgctcagcanggacgaggtgaggtttgctgtgatatttggagcaccctcaggctctgtcagaatgaccatcttcaccaggtgcacccgaaactgagcccccagggacgggtcccgaagcagttctgccccgatgttgaggttggtgagcacatagcgctctgtgtccttctggtgagcctggaagacatcggngcccacggccaccagcag

SEQ ID NO: 7 - DNA, 514 bp, Homo sapiens
ggctcagcagctgccggcggctgcagggggaccaggcgaggccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaacctagtgatatagaggaccaggtcagcatggccaggatccgtgtcgtcctcagggttgatggtctggctccacccacagacgctcagcagggacgaggtgaggttggctgtgatatttggagcaccctcaggctctgtcagaatgaccatcttcaccaggtgcacccgaaactgagcccccagggacgggtcccgaagcagttctgccccgatgttgaggttggtgagcacatagcg

SEQ ID NO: 8 - DNA, 500 bp, Homo sapiens
  misc_feature (350)..(350): n=a, c, g or t
cggctgcagggggaccaggcgaggccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaacctagtgatatagaggaccaggtcagcatggccaggatccgtgtcgtcctcagggttgatggtctggctccacccacagacgctcagcanggacgaggtgaggttggctgtgatatttggggcaccctcaggctctgtcagaatgaccatcttcaccaggtgcacccgaaactgagcccccagggacgggttccgaagcagttctgccccgatgttgaggtttgtgagcccatagcgcct

SEQ ID NO: 9 - DNA, 487 bp, Homo sapiens
gcggccgctacctgagcaggctcagcagctgccggcggctgcagggggaccaggcgaggccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaacctagtgatatagaggaccaggtcagcatggccaggatccgtgtcgtcctcagggttgatggtctggctccacccacagacgctcagcagggacgaggtgaggttggctgtgatatttggagcaccctcaggctctgtcagaatgaccatcttcaccaggtgcacccgaaactgagcccccagggacgggtc

SEQ ID NO: 10 - DNA, 535 bp, Homo sapiens
gcggccgctacctgagcaggctcagcagctgccggcggctgcagggggaccaggcgaggccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaacctagtgatatagaggaccaggtcagcatggccaggatccgtgtcgtcctcagggttgatggtctggctccacccacagacgctcagcagggacgaggtgaggttggctgtgatatttggagcaccctcaggctctgtcagaatgaccatcttcaccaggtgcacccgaaactgagccccccaggaccgggttccgaagcagttctggcccgagttgaaggtgggggagacataacgctc

SEQ ID NO: 11 - DNA, 466 bp, Homo sapiens
gcagttgccggcggctgcagggggaccaggcgaggccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaacctagtgatatagaggaccaggtcagcatggccaggatccgtgtcgtcctcagggttgatggtctggctccacccacagacgctcagcaaggacgaggtgaggttggctgtgatatttggagcaccctcaggctctgtcagaatgaccatcttcaccaggtgcacccgaaactgagtccccagggacgggtcccg

SEQ ID NO: 12 - DNA, 454 bp, Homo sapiens
cggccgctacctgagcaggctcagcagctgccggcggctgcagggggaccaggcgaggccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaacctagtgatatagaggaccaggtcagcatggccaggatccgtgtcgtcctcagggttgatggtctggctccacccacagacgctcagcagggaccaggggaggttggctgtgatatttggagcaccctcaggctctgtcagaatgaccatcttcaccagg

SEQ ID NO: 13 - DNA, 298 bp, Homo sapiens
cggccgctacctgagcaggctcagcagctgccggcggctgcagggggaccaggcgaggccggcgcggggcgcggcgccgtccgaagccatcacgtgtccgctggggccgcagccgctgccgggcgcgccgtcgtgctccaggccgaagctgtgcccaatctcatgggcaatggtgactcccaggtcgaagccagtgtcctcggtaatgaggcagctccaggttggggagcaggcaccgcccagctgggtgacgccccgcacctgccggttaccatcaggcaactccaggtcaaaccta

SEQ ID NO: 14 - DNA, 149 bp, Homo sapiens
gggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcagg

(19) United States
(12) Patent Application Publication
(10) Pub. No.: US 2004/0091965 A1
(43) Pub. Date: May 13, 2004

(54) Regulation of human adam-ts-like protein

(76) Inventor: Yonghong Xiao, Cambridge, MA (US)

Correspondence Address: BANNER & WITCOFF, 1001 G STREET N W, SUITE 1100, WASHINGTON, DC, 20001, US

(21) Appl. No.: 10/381,793

(22) PCT Filed: Sep. 26, 2001
(86) PCT No.: PCT/EP01/11124

(51) Int. Cl. C12P 21/02; C12N 5/06; C07H 21/04; C07K 14/705

(52) U.S. Cl. 435/69.1; 435/320.1; 435/325; 530/350; 536/23.5

(57) ABSTRACT

Reagents which regulate human ADAM-TS-like protein and reagents which bind to human ADAM-TS-like gene products can play a role in preventing, ameliorating, or correcting dysfunctions of diseases including, but not limited to, cadiovascular and liver disorders and COPD.

This application claims the benefit of and incorporates by reference co-pending provisional application Serial No. 60/235,881 filed Sep. 28, 2000.

### TECHNICAL FIELD OF THE INVENTION

The invention relates to the area of human ADAM-TS-like protein and its regulation.

### BACKGROUND OF THE INVENTION

ADAMs are a family of novel membrane-spanning multi-domain proteins containing a zinc metalloproteinase domain and a disintegrin domain which may serve as an integrin ligand. Hurskainen et al., J. Biol. Chem. 274, 25555-63, 1999; Kuo et al., J. Biol. Chem. 274, 18821-26, 1999; Kuno et al., J. Biol. Chem. 272, 556-62, 1997; Stone et al., J. Protein Chem. 18, 447-65, 1999; Millichip et al., Biochem. Biophys. Res. Commun. 245, 594-98, 1998. Some ADAMs are involved in the shedding and activation of cytokines and growth factors such as TNF-β. Inflammatory processes in the liver which eventually result in liver fibrosis are frequently induced by the activation of TNF-β. Other ADAMs are associated with the extracellular matrix and play other roles in inflammatory processes. For example, type IV collagenase activity is associated with some ADAMS. It is known that migration and activation of fibrogenic hepatic stellate cells is functionally linked to type IV collagenase activity. It is therefore reasonable to assume that more novel genes of this family will be detected, the products of which might be functionally involved with the induction or propagation of liver fibrosis. Therefore, novel ADAMs will be good targets for therapeutic intervention in liver fibrosis. ADAMs also may be useful for treating cardiovascular disease and COPD. There is, therefore, a continuing need in the art to identify members of this protein family which can be regulated to provide therapeutic effects.

### SUMMARY OF THE INVENTION

It is an object of the invention to provide reagents and methods of regulating a human ADAM-TS-like protein. This and other objects of the invention are provided by one or more of the embodiments described below.

One embodiment of the invention is a ADAM-TS-like protein polypeptide comprising an amino acid sequence selected from the group consisting of:

amino acid sequences which are at least about 50% identical to the amino acid sequence shown in SEQ ID NO: 2, and

the amino acid sequence shown in SEQ ID NO: 2.

Yet another embodiment of the invention is a method of screening for agents which decrease extracellular matrix degradation. A test compound is contacted with a ADAM-TS-like protein polypeptide comprising an amino acid sequence selected from the group consisting of:

amino acid sequences which are at least about 50% identical to the amino acid sequence shown in SEQ ID NO: 2, and

the amino acid sequence shown in SEQ ID NO: 2.

Binding between the test compound and the ADAM-TS-like protein polypeptide is detected. A test compound which binds to the ADAM-TS-like protein polypeptide is thereby identified as a potential agent for decreasing extracellular matrix degradation. The agent can work by decreasing the activity of the ADAM-TS-like protein.

Another embodiment of the invention is a method of screening for agents which decrease extracellular matrix degradation. A test compound is contacted with a polynucleotide encoding a ADAM-TS-like protein polypeptide, wherein the polynucleotide comprises a nucleotide sequence selected from the group consisting of:

nucleotide sequences which are at least about 50% identical to the nucleotide sequence shown in SEQ ID NO: 1, and

the nucleotide sequence shown in SEQ ID NO: 1.

Binding of the test compound to the polynucleotide is detected. A test compound which binds to the polynucleotide is identified as a potential agent for decreasing extracellular matrix degradation. The agent can work by decreasing the amount of the ADAM-TS-like protein through interacting with the ADAM-TS-like protein mRNA.

Another embodiment of the invention is a method of screening for agents which regulate extracellular matrix degradation. A test compound is contacted with a ADAM-TS-like protein polypeptide comprising an amino acid sequence selected from the group consisting of:

amino acid sequences which are at least about 50% identical to the amino acid sequence shown in SEQ ID NO: 2, and

the amino acid sequence shown in SEQ ID NO: 2.

A ADAM-TS-like protein activity of the polypeptide is detected. A test compound which increases ADAM-TS-like protein activity of the polypeptide relative to ADAM-TS-like protein activity in the absence of the test compound is thereby identified as a potential agent for increasing extracellular matrix degradation. A test compound which decreases ADAM-TS-like protein activity of the polypeptide relative to ADAM-TS-like protein activity in the absence of the test compound is thereby identified as a potential agent for decreasing extracellular matrix degradation.

Even another embodiment of the invention is a method of screening for agents which decrease extracellular matrix degradation. A test compound is contacted with a ADAM-TS-like protein product of a polynucleotide which comprises a nucleotide sequence selected from the group consisting of:

nucleotide sequences which are at least about 50% identical to the nucleotide sequence shown in SEQ ID NO: 1, and

the nucleotide sequence shown in SEQ ID NO: 1.

Binding of the test compound to the ADAM-TS-like protein product is detected. A test compound which binds to the ADAM-TS-like protein product is thereby identified as a potential agent for decreasing extracellular matrix degradation.

Still another embodiment of the invention is a method of reducing extracellular matrix degradation. A cell is contacted with a reagent which specifically binds to a polynucleotide encoding a ADAM-TS-like protein polypeptide or the product encoded by the polynucleotide, wherein the polynucleotide comprises a nucleotide sequence selected from the group consisting of:

nucleotide sequences which are at least about 50% identical to the nucleotide sequence shown in SEQ ID NO: 1, and

the nucleotide sequence shown in SEQ ID NO: 1.

ADAM-TS-like protein activity in the cell is thereby decreased.

The invention thus provides a human ADAM-TS-like protein which can be used to identify test compounds which may act, for example, as agonists or antagonists at the enzyme's active site. Human ADAM-TS-like protein and fragments thereof also are useful in raising specific antibodies which can block the enzyme and effectively reduce its activity.

### BRIEF DESCRIPTION OF THE DRAWINGS

FIG. 1 shows the DNA-sequence encoding a ADAM-TS-like protein polypeptide (SEQ ID NO: 1).

FIG. 2 shows the amino acid sequence deduced from the DNA-sequence of FIG. 1 (SEQ ID NO: 2).

FIG. 3 shows the amino acid sequence of the protein identified by Swissprot Accession No. P97857 (SEQ ID NO: 3).

FIG. 4 shows the DNA-sequence encoding a ADAM-TS-like protein polypeptide (SEQ ID NO: 4).

FIG. 5 shows the DNA-sequence encoding a ADAM-TS-like protein polypeptide (SEQ ID NO: 5).

FIG. 6 shows the DNA-sequence encoding a ADAM-TS-like protein polypeptide (SEQ ID NO: 6).

FIG. 7 shows the DNA-sequence encoding a ADAM-TS-like protein polypeptide (SEQ ID NO: 7)

FIG. 8 shows the DNA-sequence encoding a ADAM-TS-like protein polypeptide (SEQ ID NO: 8).

FIG. 9 shows the DNA-sequence encoding a ADAM-TS-like protein polypeptide (SEQ ID NO: 9).

FIG. 10 shows the DNA-sequence encoding a ADAM-TS-like protein polypeptide (SEQ ID NO: 10).

FIG. 11 shows the DNA-sequence encoding a ADAM-TS-like protein polypeptide (SEQ ID NO: 11).

FIG. 12 shows the DNA-sequence encoding a ADAM-TS-like protein polypeptide (SEQ ID NO: 12).

FIG. 13 shows the DNA-sequence encoding a ADAM-TS-like protein polypeptide (SEQ ID NO: 13).

FIG. 14 shows the DNA-sequence encoding a ADAM-TS-like protein polypeptide (SEQ ID NO: 14).

FIGS. 15A and 15B shows the BLASTP—alignment of 63_TR1 (SEQ ID NO: 2) against swiss|P97857 (SEQ ID NO: 3).

FIG. 16 shows the Prosite search result.

FIG. 17 shows the BLOCKS search results.

FIG. 18 shows the HMMPFAM—alignment of 63_TR1 against pfam|hmm|Reprolysin.

FIG. 19 shows the HMMPFAM—alignment of 63_TR1 against pfam|hmm|tsp_{—}1.

FIG. 20 shows the HMMPFAM—alignment of 63_TR1 against pfam|hmm|tsp_{—}1.

FIG. 21 shows the HMMPFAM—alignment of 63_TR1 against pfam|hmm|tsp_{—}1.

FIG. 22 shows the relative expression of ADAM-TS 1 precursor-like protein in various human tissues.

FIG. 23 shows the relative expression of ADAM-TS 1 precursor-like protein in various human respiratory tissues and cells.

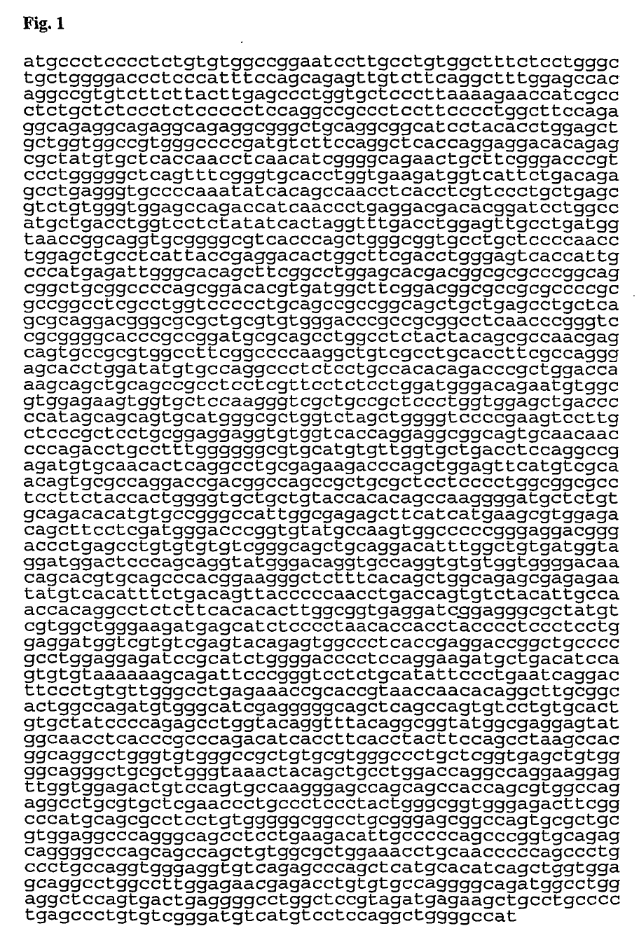

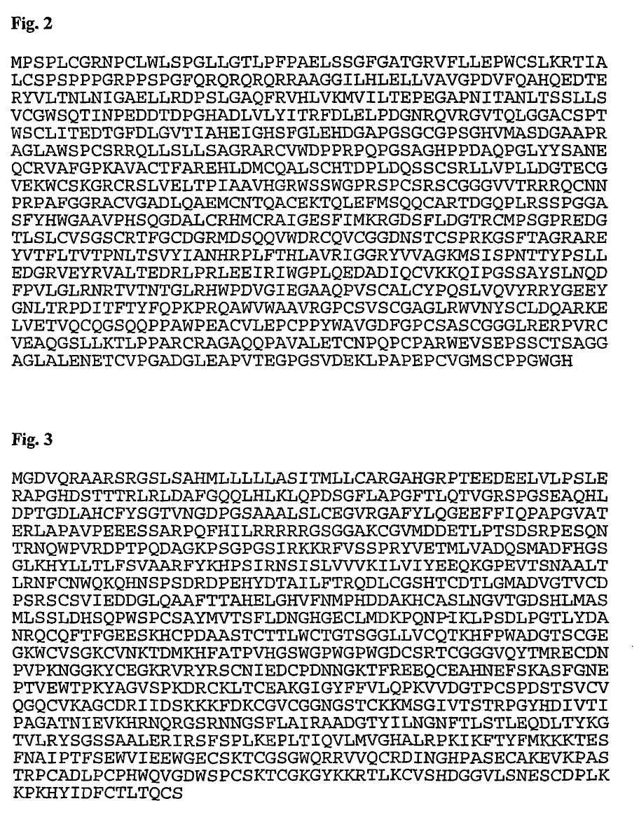

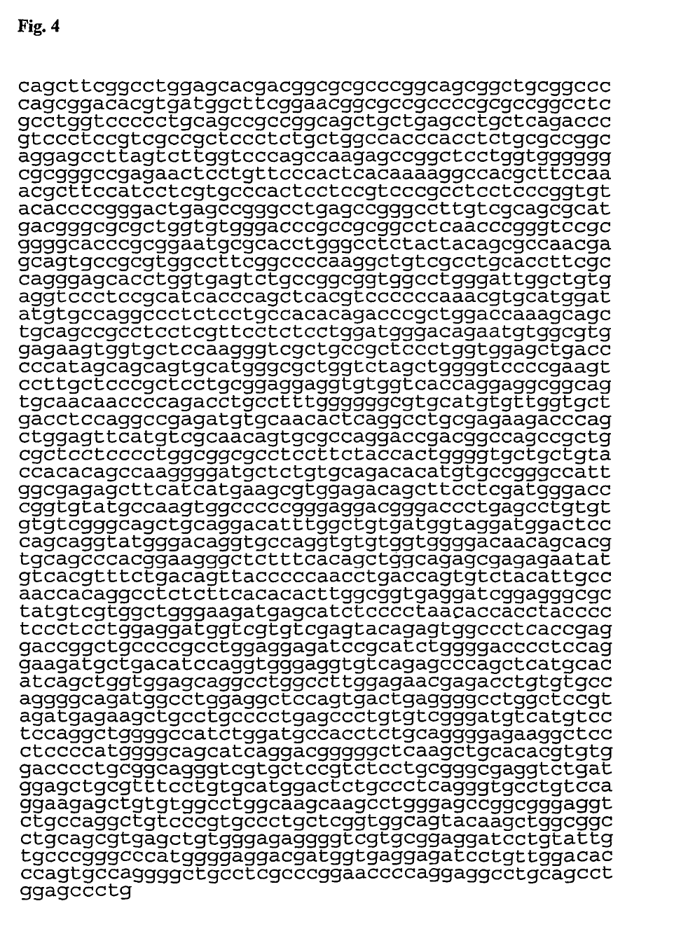

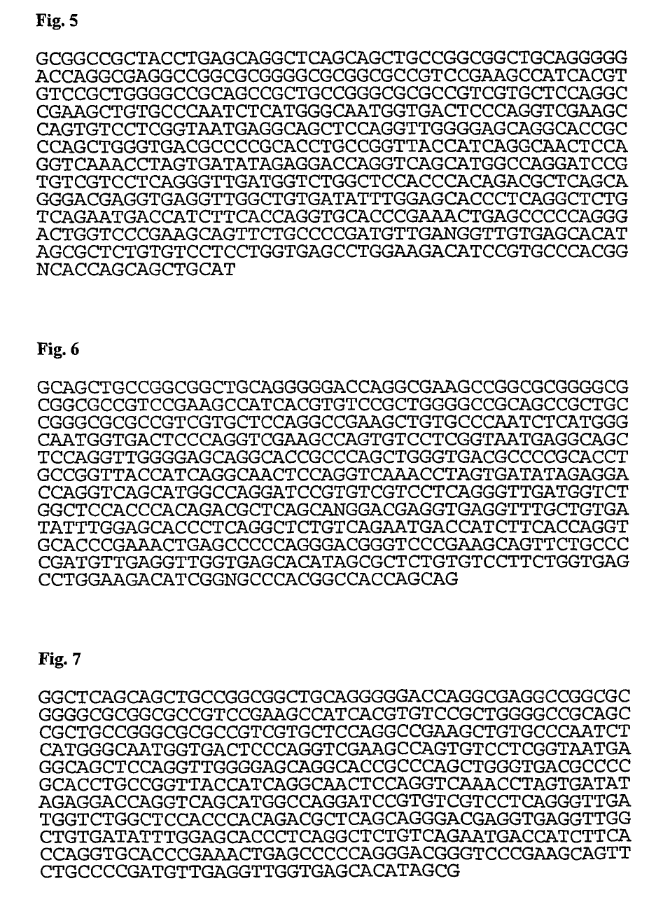

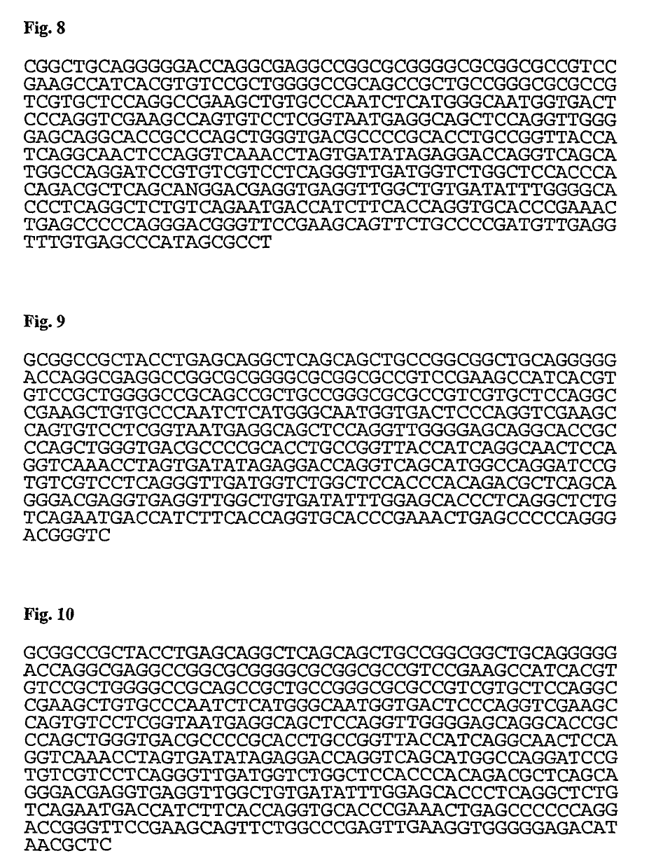

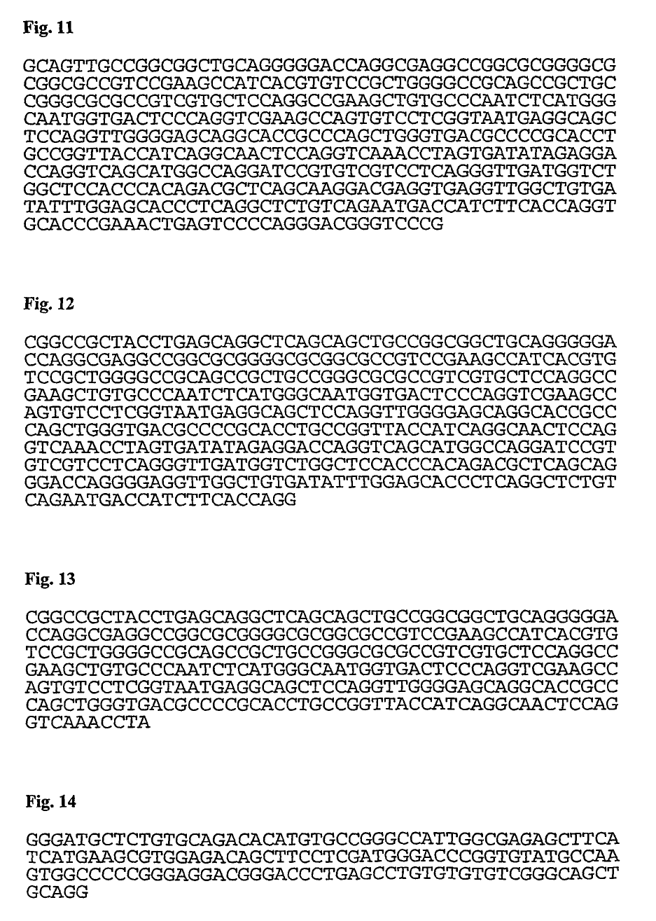

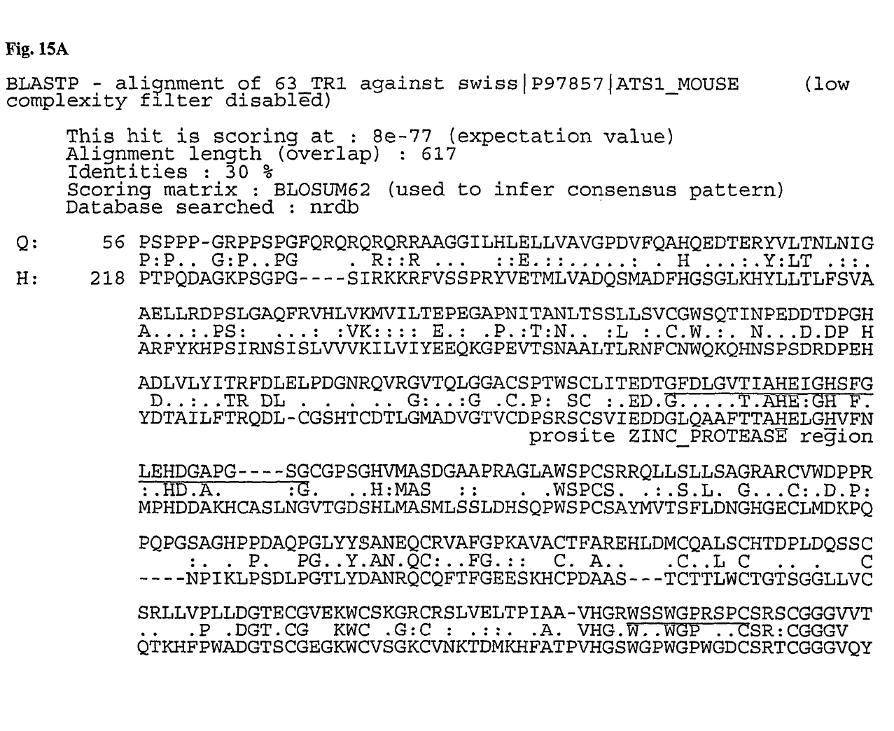

Key: HBEC=cultured human bronchial epithelial cells; H441=Clara-like cells; SMC=cultured airway smooth muscle cells; SAE=cultured small airway epithelial cells; AII=primary cultured alveolar type II cells; PMN=polymorphonuclear leukocytes; Mono=monocytes; Cult. Mono=cultured monocytes (macrophage-like).

### DETAILED DESCRIPTION OF THE INVENTION

The invention relates to an isolated polynucleotide encoding a ADAM-TS-like protein polypeptide and being selected from the group consisting of:

a) a polynucleotide encoding a ADAM-TS-like protein polypeptide comprising an amino acid sequence selected from the group consisting of:

amino acid sequences which are at least about 50% identical to the amino acid sequence shown in SEQ ID NO: 2, and

the amino acid sequence shown in SEQ ID NO: 2.

b) a polynucleotide comprising the sequence of SEQ ID NOS: 1.

c) a polynucleotide which hybridizes under stringent conditions to a polynucleotide specified in (a) and (b);

d) a polynucleotide the sequence of which deviates from the polynucleotide sequences specified in (a) to (c) due to the degeneration of the genetic code; and

e) a polynucleotide which represents a fragment, derivative or allelic variation of a polynucleotide sequence specified in (a) to (d).

Furthermore, it has been discovered by the present applicant that a novel ADAM-TS-like protein, particularly a human ADAM-TS-like protein, can be regulated to treat disorders such as cardiovascular and liver disorders and COPD. Human ADAM-TS-like protein comprises the amino acid sequence shown in SEQ ID NO: 2. Human ADAM-TS-like protein was identified by searching human sequences with the protein having the sequence shown in SEQ ID NO: 3 and identified with SwissProt Accession No. P97857.

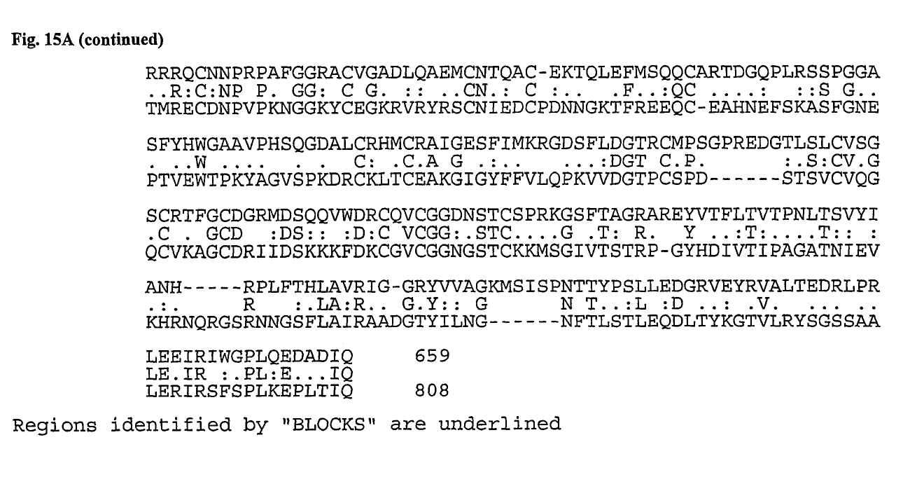

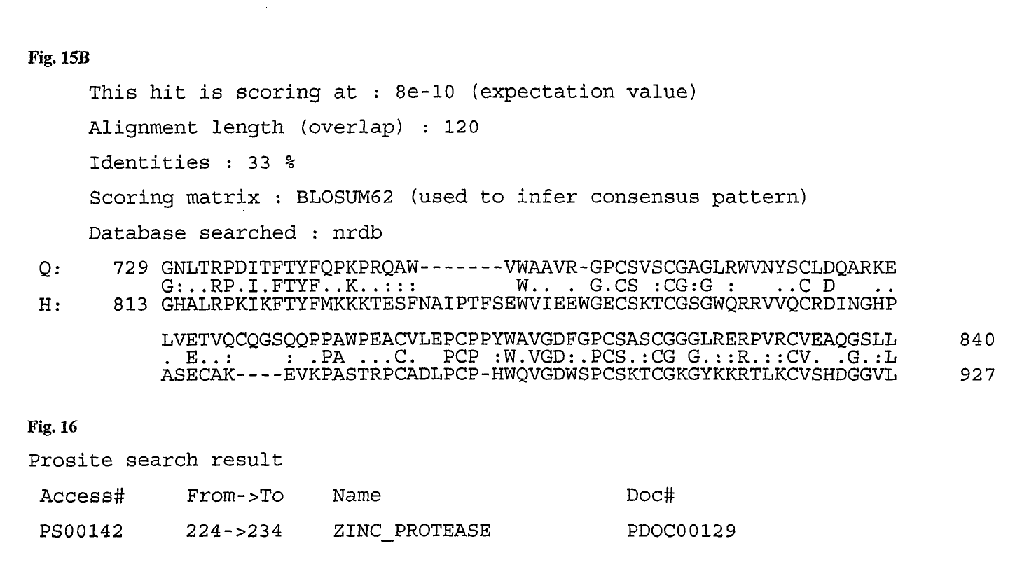

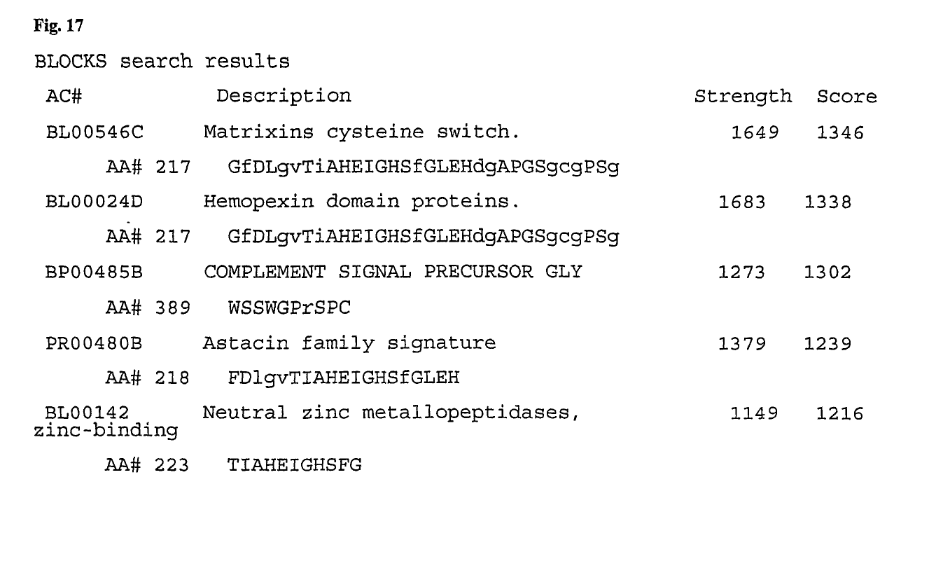

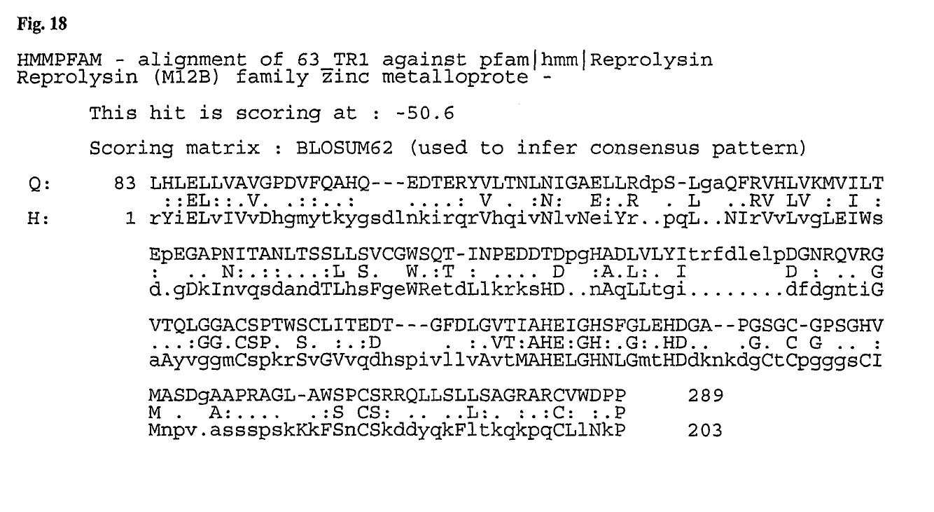

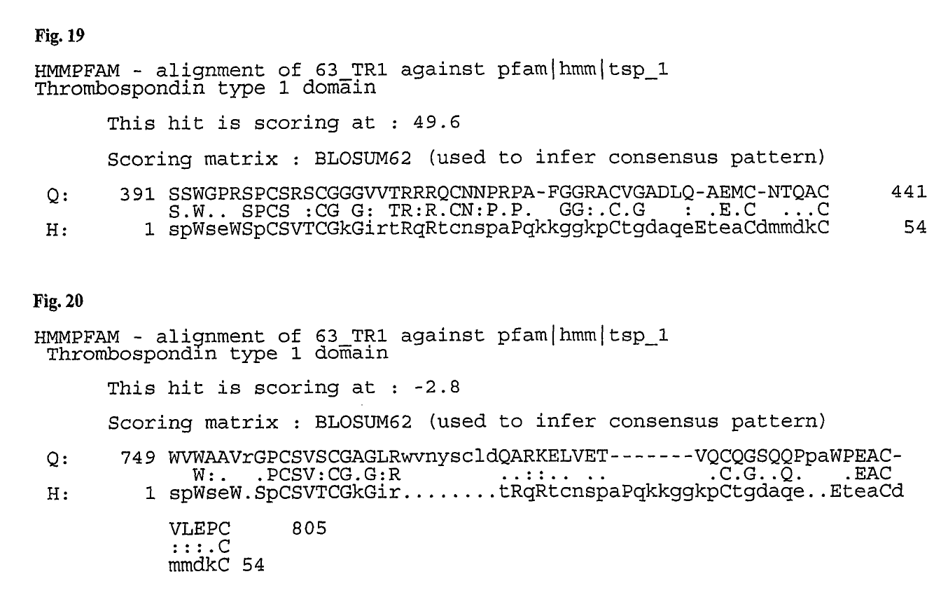

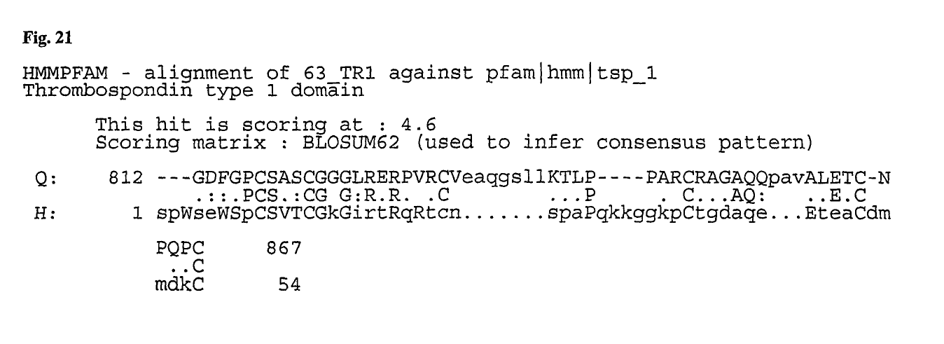

Human ADAM-TS-like protein is 30% identical over 617 amino acids and 33% identical over 120 amino acids to the protein identified with SwissProt Accession No. P97857 and annotated as “ADAM-TS 1 PRECURSOR (EC 3.4.24.-)” (FIGS. 15A and 15B). The zinc protease region is shown in bold in-FIG. 15A. Domains of human ADAM-TS-like protein are shown in FIGS. 16-21.

A coding sequence for human ADAM-TS-like protein is shown in SEQ ID NO: 1. Related ESTs (SEQ ID NOS: 4-14) are expressed in colon adenocarcinoma, pooled fetal lung, testis, B cells, and kidney tumor (metastatic to brain).

Human ADAM-TS-like protein can be used in therapeutic methods to treat disorders such as COPD, cardiovascular, and liver disorders. Human ADAM-TS 1 precursor-like protein also can be used to screen for human ADAM-TS-like protein activators and inhibitors.

Polypeptides

Human ADAM-TS-like polypeptides according to the invention comprise at least 6, 10, 15, 20, 25, 50, 75, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, or 1530 contiguous amino acids selected from the amino acid sequence shown in SEQ ID NO: 2 or a biologically active variant thereof, as defined below. A human ADAM-TS 1 precursor-like polypeptide of the invention therefore can be a portion of a human ADAM-TS-like protein, a full-length human ADAM-TS-like protein, or a fusion protein comprising all or a portion of a human ADAM-TS-like protein.

Biologically Active Variants

Human ADAM-TS-like polypeptide variants which are biologically active, e.g., has a metalloprotease activity, also are human ADAM-TS-like polypeptides. Preferably, naturally or non-naturally occurring ADAM-TS-like polypeptide variants have amino acid sequences which are at least about 50, 55, 60, 65, or 70, preferably about 75, 80, 85, 90, 96, 96, or 98% identical to the amino acid sequence shown in SEQ ID NO: 2 or a fragment thereof. Percent identity between a putative polypeptide variant and an amino acid sequence of SEQ ID NO: 2 is determined using the Blast2 alignment program (Blosum62, Expect 10, standard genetic codes).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of an ADAM-TS-like polypeptide can be found using computer programs well known in the art, such as DNASTAR software. Whether an amino acid change results in a biologically active polypeptide can readily be determined by assaying for enzyme activity. See, e.g., Black & White, Curr. Opin. Cell Biol. 10, 654-59, 1998.

Fusion Proteins

Fusion proteins are useful for generating antibodies against ADAM-TS 1 precursor-like protein amino acid sequences and for use in various assay systems. For example, fusion proteins can be used to identify proteins which interact with portions of an ADAM-TS-like polypeptide. Protein affinity chromatography or library-based assays for protein-protein interactions, such as the yeast two-hybrid or phage display systems, can be used for this purpose. Such methods are well known in the art and also can be used as drug screens.

An ADAM-TS-like protein fusion protein comprises two polypeptide segments fused together by means of a peptide bond. The first polypeptide segment comprises at least 6, 10, 15, 20, 25, 50, 75, 100, 125, 150, 175, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, or 1530 contiguous amino acids of SEQ ID NO: 2 or of a biologically active variant, such as those described above. The first polypeptide segment also can comprise full-length ADAM-TS-like protein.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horse-radish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the ADAM-TS-like polypeptide-encoding sequence and the heterologous protein sequence, so that the desired polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from the complement of SEQ ID NO: 1 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of human ADAM-TS-like polypeptide can be obtained using ADAM-TS-like polypeptide polynucleotides (described below) to make suitable probes or primers for screening cDNA expression libraries from other species, such as mice, monkeys, or yeast, identifying cDNAs which encode homologs of ADAM-TS-like polypeptide, and expressing the cDNAs as is known in the art.

Polynucleotides

An ADAM-TS-like protein polynucleotide can be single- or double-stranded and comprises a coding sequence or the complement of a coding sequence for an ADAM-TS-like polypeptide. A coding sequence for ADAM-TS 1 precursor-like protein shown in SEQ ID NO: 2 is shown in SEQ ID NO: 1.

Degenerate nucleotide sequences encoding human ADAM-TS-like polypeptides, as well as homologous nucleotide sequences which are at least about 50, 55, 60, 65, 70, preferably about 75, 90, 96, or 98% identical to the nucleotide sequence shown in SEQ ID NO: 1 or its complement also are ADAM-TS-like protein polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of ADAM-TS-like protein polynucleotides which encode biologically active ADAM-TS-like polypeptides also are ADAM-TS-like protein polynucleotides.

Identification of PolyLucleotide Variants and Homologs

Variants and homologs of the polynucleotides described above also are ADAM-TS-like protein polynucleotides. Typically, homologous polynucleotide sequences can be identified by hybridization of candidate polynucleotides to known ADAM-TS-like protein polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the ADAM-TS-like protein polynucleotides disclosed herein also can be identified by making suitable probes or primers and screening cDNA expression libraries from other species, such as mice, monkeys, or yeast. Human variants of ADAM-TS-like protein polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human ADAM-TS 1 precursor-like protein polynucleotides or ADAM-TS-like protein polynucleotides of other species can therefore be identified by hybridizing a putative homologous polynucleotide with a polynucleotide having a nucleotide sequence of SEQ ID NO: 1 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to ADAM-TS-like protein polynucleotides or their complements following stringent hybridization and/or wash conditions also are ADAM-TS-like protein polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a polynucleotide having a nucleotide sequence shown in SEQ ID NO: 1 or the complement thereof and a polynucleotide sequence which is at least about 50, 55, 60, 65, 70, preferably about 75, 90, 96, or 98% identical to one of those nucleotide sequences can be calculated, for example, using the equation of Bolton and McCarthy, Proc. Natl. Acad. Sci. U.S.A. 48,1390 (1962):

Tₘ=81.5° C.−16.6(log₁₀[Na⁺])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides

An ADAM-TS-like protein polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated ADAM-TS-like protein polynucleotides. For example, restriction enzymes and probes can be used to isolate polynucleotide fragments which comprises sphingosine kinase-like nucleotide sequences. Isolated polynucleotides are in preparations which are free or at least 70, 80, or 90% free of other molecules.

Human ADAM-TS-like protein cDNA molecules can be made with standard molecular biology techniques, using human ADAM-TS-like protein mRNA as a template. Human ADAM-TS-like protein cDNA molecules can thereafter be replicated using molecular biology techniques known in the art and disclosed in manuals such as Sambrook et al. (1989). An amplification technique, such as PCR, can be used to obtain additional copies of polynucleotides of the invention, using either human genomic DNA or cDNA as a template.

Alternatively, synthetic chemistry techniques can be used to synthesizes ADAM-TS-like protein polynucleotides. The degeneracy of the genetic code allows alternate nucleotide sequences to be synthesized which will encode a polypeptide having, for example, an amino acid sequence shown in SEQ ID NO: 2 or a biologically active variant thereof.

Extending Polynucleotides

Various PCR-based methods can be used to extend the nucleic acid sequences disclosed herein to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations also can be used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA which might be present in limited amounts in a particular sample.

Obtaining Polypeptides

Human ADAM-TS-like polypeptides can be obtained, for example, by purification from human cells, by expression of ADAM-TS-like protein polynucleotides, or by direct chemical synthesis.

Protein Purification

Human ADAM-TS-like polypeptides can be purified from any cell which expresses the enzyme, including host cells which have been transfected with ADAM-TS-like protein expression constructs. A purified ADAM-TS-like polypeptide is separated from other compounds which normally associate with the ADAM-TS-like polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified ADAM-TS-like polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express a human ADAM-TS-like protein polynucleotide, the polynucleotide can be inserted into an expression vector which contains the necessary elements for the transcription and translation of the inserted coding sequence. Methods which are well known to those skilled in the art can be used to construct expression vectors containing sequences encoding ADAM-TS-like polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding an ADAM-TS-like polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding an ADAM-TS-like polypeptide, vectors based on SV40 or EBV can be used with an appropriate selectable marker.

Bacterial and Yeast Expression Systems

In bacterial systems, a number of expression vectors can be selected depending upon the use intended for the ADAM-TS-like polypeptide. For example, when a large quantity of a polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the polypeptide can be ligated into the vector in frame with sequences for the amino-terminal Met and the subsequent 7 residues of β-galactosidase so that a hybrid protein is produced. pIN vectors (Van Heeke & Schuster, J. Biol. Chem. 264, 5503-5509, 1989) or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see Ausubel et al. (1989) and Grant et al., Methods Enzymol. 153, 516-544, 1987.

Plant and Insect Expression Systems

If plant expression vectors are used, the expression of sequences encoding ADAM-TS-like polypeptides can be driven by any of a number of promoters. For example, viral promoters such as the ³⁵S and 19S promoters of CaMV can be used alone or in combination with the omega leader sequence from TMV (Takamatsu, EMBO J. 6, 307-311, 1987). Alternatively, plant promoters such as the small subunit of RUBISCO or heat shock promoters can be used (Coruzzi et al., EMBO J. 3, 1671-1680, 1984; Broglie et al., Science 224, 838-843, 1984; Winter et al., Results Probl. Cell Differ. 17, 85-105, 1991). These constructs can be introduced into plant cells by direct DNA transformation or by pathogen-mediated transfection. Such techniques are described in a number of generally available reviews (e.g., Hobbs or Murray, in McGRAW HILL YEARBOOK OF SCIENCE AND TECHNOLOGY, McGraw Hill, New York, N.Y., pp. 191-196, 1992).

An insect system also can be used to express an ADAM-TS-like polypeptide. For example, in one such system Autographa californica nuclear polyhedrosis virus (AcNPV) is used as a vector to express foreign genes in Spodoptera frugiperda cells or in Trichoplusia larvae. Sequences encoding ADAM-TS-like polypeptides can be cloned into a non-essential region of the virus, such as the polyhedrin gene, and placed under control of the polyhedrin promoter. Successful insertion of ADAM-TS-like polypeptides will render the polyhedrin gene inactive and produce recombinant virus lacking coat protein. The recombinant viruses can then be used to infect S. frugiperda cells or Trichoplusia larvae in which ADAM-TS-like polypeptides can be expressed (Engelhard et al., Proc. Nat. Acad. Sci 91, 3224-3227, 1994).

Mammalian Expression Systems

A number of viral-based expression systems can be used to express ADAM-TS-like polypeptides in mammalian host cells. For example, if an adenovirus is used as an expression vector, sequences encoding ADAM-TS-like polypeptides can be ligated into an adenovirus transcription/translation complex comprising the late promoter and tripartite leader sequence. Insertion in a non-essential E1 or E3 region of the viral genome can be used to obtain a viable virus which is capable of expressing an ADAM-TS-like polypeptide in infected host cells (Logan & Shenk, Proc. Natl. Acad. Sci. 81, 3655-3659, 1984). If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding ADAM-TS-like polypeptides. Such signals include the ATG initiation codon and adjacent sequences. In cases where sequences encoding an ADAM-TS-like polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20, 125-162, 1994).

Host Cells

A host cell strain can be chosen for its ability to modulate the expression of the inserted sequences or to process the expressed ADAM-TS-like polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express ADAM-TS-like polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced ADAM-TS-like protein sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines. These include, but are not limited to, the herpes simplex virus thymidine kinase (Wigler et al., Cell 11, 223-32, 1977) and adenine phosphoribosyltransferase (Lowy et al., Cell 22, 817-23, 1980) genes which can be employed in tk⁻ or aprt⁻ cells, respectively. Also, antimetabolite, antibiotic, or herbicide resistance can be used as the basis for selection. For example, dhfr confers resistance to methotrexate (Wigler et al., Proc. Natl. Acad. Sci. 77, 3567-70, 1980), npt confers resistance to the aminoglycosides, neomycin and G-418 (Colbere-Garapin et al., J. Mol. Biol. 150, 1-14, 1981), and als and pat confer resistance to chlorsulfuron and phosphinotricin acetyltransferase, respectively (Murray, 1992, supra). Additional selectable genes have been described. For example, trpB allows cells to utilize indole in place of tryptophan, or hisD, which allows cells to utilize histinol in place of histidine (Hartman & Mulligan, Proc. Natl. Acad. Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Detecting Expression

Although the presence of marker gene expression suggests that the ADAM-TS-like protein polynucleotide is also present, its presence and expression may need to be confirmed. For example, if a sequence encoding an ADAM-TS-like polypeptide is inserted within a marker gene sequence, transformed cells containing sequences which encode an ADAM-TS-like polypeptide can be identified by the absence of marker gene function. Alternatively, a marker gene can be placed in tandem with a sequence encoding an ADAM-TS-like polypeptide under the control of a single promoter. Expression of the marker gene in response to induction or selection usually indicates expression of the ADAM-TS-like protein polynucleotide.

Alternatively, host cells which contain an ADAM-TS-like protein polynucleotide and which express an ADAM-TS-like polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding an ADAM-TS-like polypeptide can be detected by DNA-DNA or DNA-RNA hybridization or amplification using probes or fragments or fragments of polynucleotides encoding an ADAM-TS-like polypeptide. Nucleic acid amplification-based assays involve the use of oligonucleotides selected from sequences encoding an ADAM-TS-like polypeptide to detect transformants which contain an ADAM-TS-like protein polynucleotide.

A variety of protocols for detecting and measuring the expression of an ADAM-TS-like polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on an ADAM-TS-like polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158, 1211-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding ADAM-TS-like polypeptides include oligolabeling, nick translation, end-labeling, or PCR amplification using a labeled nucleotide. Alternatively, sequences encoding an ADAM-TS-like polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Polypeptides

Host cells transformed with nucleotide sequences encoding an ADAM-TS-like polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode ADAM-TS-like polypeptides can be designed to contain signal sequences which direct secretion of soluble ADAM-TS-like polypeptides through a prokaryotic or eukaryotic cell membrane or which direct the membrane insertion of membrane-bound ADAM-TS-like polypeptide.

As discussed above, other constructions can be used to join a sequence encoding an ADAM-TS-like polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase (Invitrogen, San Diego, Calif.) between the purification domain and the ADAM-TS-like polypeptide also can be used to facilitate purification. One such expression vector provides for expression of a fusion protein containing an ADAM-TS-like polypeptide and 6 histidine residues preceding a thioredoxin or an enterokinase cleavage site. The histidine residues facilitate purification by IMAC (immobilized metal ion affinity chromatography, as described in Porath et al., Prot. Exp. Purif. 3, 263-281, 1992), while the enterokinase cleavage site provides a means for purifying the ADAM-TS 1 precursor-like polypeptide from the fusion protein. Vectors which contain fusion proteins are disclosed in Kroll et al., DNA Cell Biol. 12, 441-453, 1993.

Chemical Synthesis

Sequences encoding an ADAM-TS-like polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, an ADAM-TS-like polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of ADAM-TS-like polypeptides can be separately synthesized and combined using chemical methods to produce a full-length molecule.

The newly synthesized peptide can be substantially purified by preparative high performance liquid chromatography (e.g., Creighton, PROTEINS: STRUCTURES AND MOLECULAR PRINCIPLES, WH Freeman and Co., New York, N.Y., 1983). The composition of a synthetic ADAM-TS-like polypeptide can be confirmed by amino acid analysis or sequencing (e.g., the Edman degradation procedure; see Creighton, supra). Additionally, any portion of the amino acid sequence of the ADAM-TS-like polypeptide can be altered during direct synthesis and/or combined using chemical methods with sequences from other proteins to produce a variant polypeptide or a fusion protein.

Production of Altered Polypeptides

As will be understood by those of skill in the art, it may be advantageous to produce ADAM-TS-like polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter ADAM-TS-like polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of an ADAM-TS-like polypeptide. “Antibody” as used herein includes intact immunoglobulin molecules, as well as fragments thereof, such as Fab, F(ab′)₂, and Fv, which are capable of binding an epitope of an ADAM-TS-like polypeptide. Typically, at least 6, 8, 10, or 12 contiguous amino acids are required to form an epitope. However, epitopes which involve non-contiguous amino acids may require more, e.g, at least 15, 25, or 50 amino acids.

An antibody which specifically binds to an epitope of an ADAM-TS 1 precursor-like polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to an ADAM-TS-like polypeptide provides a detection signal at least 5-, 10-, or 20-fold higher than a detection signal provided with other proteins when used in an immunochemical assay. Preferably, antibodies which specifically bind to ADAM-TS-like polypeptides do not detect other proteins in immunochemical assays and can immunoprecipitate an ADAM-TS-like polypeptide from solution.

Human ADAM-TS-like polypeptides can be used to immunize a mammal, such as a mouse, rat, rabbit, guinea pig, monkey, or human, to produce polyclonal antibodies. If desired, an ADAM-TS-like polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calnette-Guerin) and Corynebacteriun parvuim are especially useful.

Monoclonal antibodies which specifically bind to an ADAM-TS-like polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methods 81, 31-42, 1985; Cote et al., Proc. Natl. Acad. Sci. 80, 2026-2030, 1983; Cole et al., Mol. Cell Biol. 62, 109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to an ADAM-TS-like polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to ADAM-TS-like polypeptides. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol, 15, 159-63. Construction of bivalent, bispecific single-chain antibodies is taught in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., 1995, Int. J. Cancer 61, 497-501; Nicholls et al., 1993, J. Immunol. Meth. 165, 81-91).

Antibodies which specifically bind to ADAM-TS-like polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which an ADAM-TS-like polypeptide is bound. The bound antibodies can then be eluted from the column using a buffer with a high salt concentration.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of ADAM-TS-like gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583,1990.

Modifications of ADAM-TS-like gene expression can be obtained by designing antisense oligonucleotides which will form duplexes to the control, 5′, or regulatory regions of the ADAM-TS-like gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC

APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of an ADAM-TS-like protein polynucleotide. Antisense oligonucleotides which comprise, for example, 2, 3, 4, or 5 or more stretches of contiguous nucleotides which are precisely complementary to an ADAM-TS-like protein polynucleotide, each separated by a stretch of contiguous nucleotides which are not complementary to adjacent ADAM-TS-like protein nucleotides, can provide sufficient targeting specificity for ADAM-TS-like protein mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular ADAM-TS-like protein polynucleotide sequence.

Antisense oligonucleotides can be modified without affecting their ability to hybridize to an ADAM-TS-like protein polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of an ADAM-TS-like protein polynucleotide can be used to generate ribozymes which will specifically bind to mRNA transcribed from the ADAM-TS-like protein polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within an ADAM-TS-like protein RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate ADAM-TS 1 precursor-like protein RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease ADAM-TS-like protein expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Differentially Expressed Genes

Described herein are methods for the identification of genes whose products interact with human ADAM-TS-like protein. Such genes may represent genes which are differentially expressed in disorders including, but not limited to, COPD, cardiovascular, and liver disorders. Further, such genes may represent genes which are differentially regulated in response to manipulations relevant to the progression or treatment of such diseases. Additionally, such genes may have a temporally modulated expression, increased or decreased at different stages of tissue or organism development. A differentially expressed gene may also have its expression modulated under control versus experimental conditions. In addition, the human choline/ethanolamine 1 precursor-like gene or gene product may itself be tested for differential expression.

The degree to which expression differs in a normal versus a diseased state need only be large enough to be visualized via standard characterization techniques such as differential display techniques. Other such standard characterization techniques by which expression differences may be visualized include but are not limited to, quantitative RT (reverse transcriptase), PCR, and Northern analysis.

Identification of Differentially Expressed Genes

To identify differentially expressed genes total RNA or, preferably, mRNA is isolated from tissues of interest. For example, RNA samples are obtained from tissues of experimental subjects and from corresponding tissues of control subjects. Any RNA isolation technique which does not select against the isolation of mRNA may be utilized for the purification of such RNA samples. See, for example, Ausubel et al., ed., CURRENT PROTOCOLS IN MOLECULAR BIOLOGY, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples which represent RNA produced by differentially expressed genes are identified by methods well known to those of skill in the art. They include, for example, differential screening (Tedder et al., Proc. Natl. Acad. Sci. U.S.A. 85, 208-12, 1988), subtractive hybridization (Hedrick et al., Nature 308, 149-53; Lee et al., Proc. Natl. Acad. Sci. U.S.A. 88, 2825, 1984), and, preferably, differential display (Liang & Pardee, Science 257, 967-71, 1992; U.S. Pat. No. 5,262,311).

The differential expression information may itself suggest relevant methods for the treatment of disorders involving the human ADAM-TS-like protein. For example, treatment may include a modulation of expression of the differentially expressed genes and/or the gene encoding the human ADAM-TS-like protein. The differential expression information may indicate whether the expression or activity of the differentially expressed gene or gene product or the human 1 precursor-like gene or gene product are up-regulated or down-regulated.

Screening Methods

The invention provides assays for screening test compounds which bind to or modulate the activity of an ADAM-TS-like polypeptide or an ADAM-TS-like protein polynucleotide. A test compound preferably binds to an ADAM-TS-like polypeptide or polynucleotide. More preferably, a test compound decreases or increases ADAM-TS-like protein by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound”—library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to ADAM-TS 1 precursor-like polypeptides or polynucleotides or to affect ADAM-TS-like protein activity or ADAM-TS-like gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule which binds to and occupies, for example, the active site of the ADAM-TS-like polypeptide, such that normal biological activity is prevented. Examples of such small molecules include, but are not limited to, small peptides or peptide-like molecules.

In binding assays, either the test compound or the ADAM-TS-like polypeptide can comprise a detectable label, such as a fluorescent, radioisotopic, chemiluminescent, or enzymatic label, such as horseradish peroxidase, alkaline phosphatase, or luciferase. Detection of a test compound which is bound to the ADAM-TS-like polypeptide can then be accomplished, for example, by direct counting of radio-emmission, by scintillation counting, or by determining conversion of an appropriate substrate to a detectable product.

Alternatively, binding of a test compound to an ADAM-TS-like polypeptide can be determined without labeling either of the interactants. For example, a microphysiometer can be used to detect binding of a test compound with an ADAM-TS-like polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and an ADAM-TS-like polypeptide (McConnell et al., Science 257, 1906-1912, 1992).

Determining the ability of a test compound to bind to an ADAM-TS 1 precursor-like polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, an ADAM-TS-like polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent WO94/10300), to identify other proteins which bind to or interact with the ADAM-TS-like polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding an ADAM-TS-like polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the ADAM-TS-like polypeptide.

It may be desirable to immobilize either the ADAM-TS-like polypeptide (or polynucleotide) or the test compound to facilitate separation of bound from unbound forms of one or both of the interactants, as well as to accommodate automation of the assay. Thus, either the ADAM-TS-like polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the enzyme polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a v enzyme polypeptide (or polynucleotide) can be accomplished in any vessel suitable for containing the reactants. Examples of such vessels include microtiter plates, test tubes, and microcentrifuge tubes.

In one embodiment, the ADAM-TS-like polypeptide is a fusion protein comprising a domain that allows the ADAM-TS-like polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed ADAM-TS-like polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either an ADAM-TS-like polypeptide (or polynucleotide) or a test compound can be immobilized utilizing conjugation of biotin and streptavidin. Biotinylated ADAM-TS-like polypeptides (or polynucleotides) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to an ADAM-TS-like polypeptide, polynucleotide, or a test compound, but which do not interfere with a desired binding site, such as the active site of the enzyme, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the ADAM-TS-like polypeptide or test compound, enzyme-linked assays which rely on detecting an activity of the ADAM-TS-like polypeptide, and SDS gel electrophoresis under non-reducing conditions.

Screening for test compounds which bind to an ADAM-TS-like polypeptide or polynucleotide also can be carried out in an intact cell. Any cell which comprises an ADAM-TS-like polypeptide or polynucleotide can be used in a cell-based assay system. An ADAM-TS-like protein polynucleotide can be naturally occurring in the cell or can be introduced using techniques such as those described above. Binding of the test compound to an ADAM-TS-like polypeptide or polynucleotide is determined as described above.

Enzyme Assays

Test compounds can be tested for the ability to increase or decrease the metalloprotease activity of a human ADAM-TS-like polypeptide. Metalloprotease activity can be measured as is known in the art. See, e.g., Black & White, Curr. Opin. Cell Biol. 10, 654-59, 1998.

Enzyme assays can be carried out after contacting either a purified ADAM-TS-like polypeptide, a cell membrane preparation, or an intact cell with a test compound. A test compound which decreases activity of an ADAM-TS-like polypeptide by at least about 10, preferably about 50, more preferably about 75, 90, or 100% is identified as a potential therapeutic agent for decreasing ADAM-TS 1 precursor-like protein activity. A test compound which increases activity of a human ADAM-TS-like polypeptide by at least about 10, preferably about 50, more preferably about 75, 90, or 100% is identified as a potential therapeutic agent for increasing human ADAM-TS-like protein activity.

Gene Expression

In another embodiment, test compounds which increase or decrease ADAM-TS-like gene expression are identified. An ADAM-TS-like protein polynucleotide is contacted with a test compound, and the expression of an RNA or polypeptide product of the polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of enzyme mRNA or polypeptide expression in the cells can be determined by methods well known in the art for detecting mRNA or polypeptide. Either qualitative or quantitative methods can be used. The presence of polypeptide products of an ADAM-TS-like protein polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into an ADAM-TS-like polypeptide.

Such screening can be carried out either in a cell-free assay system or in an intact cell. Any cell which expresses an ADAM-TS-like protein polynucleotide can be used in a cell-based assay system. The ADAM-TS-like protein polynucleotide can be naturally occurring in the cell or can be introduced using techniques such as those described above. Either a primary culture or an established cell line, such as CHO or human embryonic kidney 293 cells, can be used.

Pharmaceutical Compositions

The invention also provides pharmaceutical compositions which can be administered to a patient to achieve a therapeutic effect. Pharmaceutical compositions of the invention can comprise, for example, an ADAM-TS-like polypeptide, ADAM-TS-like protein polynucleotide, ribozymes or antisense oligonucleotides, antibodies which specifically bind to an ADAM-TS-like polypeptide, or mimetics, agonists, antagonists, or inhibitors of an ADAM-TS 1 precursor-like polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCINCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

Human ADAM-TS-like protein can be regulated to treat liver disorders. Some ADAMs are involved in the shedding and activation of cytokines and growth factors such as TNF-β. Inflammatory processes in the liver which eventually result in liver fibrosis are frequently induced by the activation of TNF-β. Other ADAMs are associated with the extracellular matrix and play other roles in inflammatory processes. For example, type IV collagenase activity is associated with some ADAMS. It is known that migration and activation of fibrogenic hepatic stellate cells is functionally linked to type IV collagenase activity. It is therefore reasonable to assume that more novel genes of this family will be detected, the products of which might be functionally involved with the induction or propagation of liver fibrosis. Therefore, novel ADAMs will be good targets for therapeutic intervention in liver fibrosis.

ADAM-TS-like protein also can be regulated to treat cardiovascular disorders. Cardiovascular diseases include the following disorders of the heart and the vascular system: congestive heart failure, myocardial infarction, ischemic diseases of the heart, all kinds of atrial and ventricular arrhythmias, hypertensive vascular diseases, and peripheral vascular diseases.

Heart failure is defined as a pathophysiologic state in which an abnormality of cardiac function is responsible for the failure of the heart to pump blood at a rate commensurate with the requirement of the metabolizing tissue. It includes all forms of pumping failure, such as high-output and low-output, acute and chronic, right-sided or left-sided, systolic or diastolic, independent of the underlying cause.

Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included, as well as the acute treatment of MI and the prevention of complications.

Ischemic diseases are conditions in which the coronary flow is restricted resulting in a perfusion which is inadequate to meet the myocardial requirement for oxygen. This group of diseases includes stable angina, unstable angina, and asymptomatic ischemia.

Arrhythmias include all forms of atrial and ventricular tachyarrhythmias (atrial tachycardia, atrial flutter, atrial fibrillation, atrio-ventricular reentrant tachycardia, preexcitation syndrome, ventricular tachycardia, ventricular flutter, and ventricular fibrillation), as well as bradycardic forms of arrhythmias.

Hypertensive vascular diseases include primary as well as all kinds of secondary arterial hypertension (renal, endocrine, neurogenic, others). The disclosed gene and its product may be used as drug targets for the treatment of hypertension as well as for the prevention of all complications.

Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon, and venous disorders.

ADAM-TS-like protein also can be regulated to treat chronic obstructive pulmonary disease. Chronic obstructive pulmonary (or airways) disease (COPD) is a condition defined physiologically as airflow obstruction that generally results from a mixture of emphysema and peripheral airway obstruction due to chronic bronchitis (Senior & Shapiro, Pulmonary Diseases and Disorders, 3d ed., New York, McGraw-Hill, 1998, pp. 659-681, 1998; Barnes, Chest 117, 10S-14S, 2000). Emphysema is characterized by destruction of alveolar walls leading to abnormal enlargement of the air spaces of the lung. Chronic bronchitis is defined clinically as the presence of chronic productive cough for three months in each of two successive years. In COPD, airflow obstruction is usually progressive and is only partially reversible. By far the most important risk factor for development of COPD is cigarette smoking, although the disease does occur in non-smokers.

Chronic inflammation of the airways is a key pathological feature of COPD (Senior & Shapiro, 1998). The inflammatory cell population comprises increased numbers of macrophages, neutrophils, and CD8⁺ lymphocytes. Inhaled irritants, such as cigarette smoke, activate macrophages which are resident in the respiratory tract, as well as epithelial cells leading to release of chemokines (e.g., interleukin-8) and other chemotactic factors. These chemotactic factors act to increase the neutrophil/monocyte trafficking from the blood into the lung tissue and airways. Neutrophils and monocytes recruited into the airways can release a variety of potentially damaging mediators such as proteolytic enzymes and reactive oxygen species. Matrix degradation and emphysema, along with airway wall thickening, surfactant dysfunction, and mucus hypersecretion, all are potential sequelae of this inflammatory response that lead to impaired airflow and gas exchange.

COPD is characterized by damage to the lung extracellular matrix and emphysema can be viewed as the pathologic process that affects the lung parenchyma. This process eventually leads to the destruction of the airway walls resulting in permanent airspace enlargement (Senior and Shapiro, in PULMONARY DISEASES AND DISORDERS, 3^{rd} ed., New York, McGraw-Hill, 1998, pp. 659-681, 1998). The observation that inherited deficiency of al-antitrypsin (al-AT), the primary inhibitor of neutrophil elastase, predisposes individuals to early onset emphysema, and that intrapulmonary instillation of elastolytic enzymes in experimental animals causes emphysema, led to the elastase:antielastase hypothesis for the pathogenesis of emphysema (Eriksson, Acta Med. Scand. 177(Suppl.), 432, 1965, Gross, J. Occup. Med. 6, 481-84, 1964). This in turn led to the concept that destruction of elastin in the lung parenchyma is the basis of the development of emphysema.

A broad range of immune and inflammatory cells including neutrophils, macrophages, T lymphocytes and eosinophils contain proteolytic enzymes that could contribute to the destruction of lung extracellular matrix (Shapiro, 1999). In addition, a number of different classes of proteases have been identified that have the potential to contribute to lung matrix destruction. These include serine proteases, matrix metalloproteinases and cysteine proteases. Of these classes of enzymes, a number can hydrolyze elastin and have been shown to be elevated in COPD patients (neutrophil elastase, MMP-2, 9, 12) (Culpitt et al., Am. J. Respir. Crit. Care Med. 160, 1635-39, 1999, Shapiro, Am. J. Crit. Care Med. 160 (5), S29-S32,1999).

It is expected that in the future novel members of the existing classes of proteases and new classes of proteases will be identified that play a significant role in the damage of the extracellular lung matrix including elastin proteolysis. Novel protease targets therefore remain very attractive therapeutic targets.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or an ADAM-TS-like polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects ADAM-TS-like protein activity can be administered to a human cell, either in vitro or in vivo, to reduce ADAM-TS 1 precursor-like protein activity. The reagent preferably binds to an expression product of a human ADAM-TS-like gene. If the expression product is a protein, the reagent is preferably an antibody. For treatment of human cells ex vivo, an antibody can be added to a preparation of stem cells which have been removed from the body. The cells can then be replaced in the same or another human body, with or without tonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 mmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 mmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE

TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

Determination of a Therapeutically Effective Dose

The determination of a therapeutically effective dose is well within the capability of those skilled in the art. A therapeutically effective dose refers to that amount of active ingredient which increases or decreases kinase-like enzyme activity relative to the ADAM-TS-like protein activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can J be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of an ADAM-TS-like gene or the activity of an ADAM-TS-like polypeptide by at least about 10, preferably about 50, more preferably about 75, 90, or 100% relative to the absence of the reagent. The effectiveness of the mechanism chosen to decrease the level of expression of an ADAM-TS-like gene or the activity of an ADAM-TS-like polypeptide can be assessed using methods well known in the art, such as hybridization of nucleotide probes to ADAM-TS-like protein-specific mRNA, quantitative RT-PCR, immunologic detection of an ADAM-TS-like polypeptide, or measurement of ADAM-TS-like protein activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

Human ADAM-TS-like protein also can be used in diagnostic assays for detecting diseases and abnormalities or susceptibility to diseases and abnormalities related to the presence of mutations in the nucleic acid sequences which encode the enzyme. For example, differences can be determined between the cDNA or genomic sequence encoding ADAM-TS-like protein in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl. Acad. Sci. USA 85, 4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of an ADAM-TS-like protein also can be detected in various tissues. Assays used to detect levels of the receptor polypeptides in a body sample, such as blood or a tissue biopsy, derived from a host are well known to those of skill in the art and include radioimmunoassays, competitive binding assays, Western blot analysis, and ELISA assays.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention.

### EXAMPLE 1

Detection of ADAM-TS-Like Protein Activity

The polynucleotide of SEQ ID NO: 1 is inserted into the expression vector pCEV4 and the expression vector pCEV4-ADAM-TS-like protein polypeptide obtained is transfected into human embryonic kidney 293 cells. From these cells extracts are obtained and for determing collagenase activity, 5 μl of sample is added to 45 μl of 10 mg-mll gelatin or 10 mg-mll type IV collagen (Sigma type IV) in a solution containing 50 mM Tris Cl, 145 mM NaCl, and 5 mM CaCl₂ and incubated at 37° C. for 18 h. Intact collagen is precipitated with an equal volume of 50% trichloroacetic acid, and the levels of soluble amino acids and peptides are estimated by a standard ninhydrin method (16). Activity is measured as the difference in acid-soluble amino acids and peptides between the 18-h samples and zero time controls in which 5 μl of sample is added to substrate after it has been incubated at 37° C. for 18 h and immediately prior to acid precipitation. Activity is estimated by comparison with a standard curve of clostridiopeptidase A (EC 3.4.24.3; Sigma) assuming a preparation activity of 1.8 U mgl as labeled. It is shown that the polypeptide of SEQ ID NO: 2 has a ADAM-TS-like protein activity.

### EXAMPLE 2

Expression of Recombinant Human ADAM-TS-Like Protein

The Pichia pastoris expression vector pPICZB (Invitrogen, San Diego, Calif.) is used to produce large quantities of recombinant human ADAM-TS-like polypeptides in yeast. The ADAM-TS-like protein-encoding DNA sequence is derived from SEQ ID NO: 1. Before insertion into vector pPICZB, the DNA sequence is modified by well known methods in such a way that it contains at its 5′-end an initiation codon and at its 3′-end an enterokinase cleavage site, a His6 reporter tag and a termination codon. Moreover, at both termini recognition sequences for restriction endonucleases are added and after digestion of the multiple cloning site of pPICZ B with the corresponding restriction enzymes the modified DNA sequence is ligated into pPICZB. This expression vector is designed for inducible expression in Pichia pastoris, driven by a yeast promoter. The resulting pPICZ/md-His6 vector is used to transform the yeast.

The yeast is cultivated under usual conditions in 5 liter shake flasks and the recombinantly produced protein isolated from the culture by affinity chromatography (Ni-NTA-Resin) in the presence of 8 M urea. The bound polypeptide is eluted with buffer, pH 3.5, and neutralized. Separation of the polypeptide from the His6 reporter tag is accomplished by site-specific proteolysis using enterokinase (Invitrogen, San Diego, Calif.) according to manufacturer's instructions. Purified human ADAM-TS-like polypeptide is obtained.

### EXAMPLE 3

Identification of Test Compounds That Bind to ADAM-TS-Like Polypeptides

Purified ADAM-TS-like polypeptides comprising a glutathione-S— transferase protein and absorbed onto glutathione-derivatized wells of 96-well microtiter plates are contacted with test compounds from a small molecule library at pH 7.0 in a physiological buffer solution. Human ADAM-TS-like polypeptides comprise the amino acid sequence shown in SEQ ID NO: 2. The test compounds comprise a fluorescent tag. The samples are incubated for 5 minutes to one hour. Control samples are incubated in the absence of a test compound.

The buffer solution containing the test compounds is washed from the wells. Binding of a test compound to an ADAM-TS-like polypeptide is detected by fluorescence measurements of the contents of the wells. A test compound which increases the fluorescence in a well by at least 15% relative to fluorescence of a well in which a test compound is not incubated is identified as a compound which binds to an ADAM-TS-like polypeptide.

### EXAMPLE 4

Identification of a Test Compound Which Decreases ADAM-TS-Like Gene Expression

A test compound is administered to a culture of human cells transfected with an ADAM-TS-like protein expression construct and incubated at 37° C. for 10 to 45 minutes. A culture of the same type of cells which have not been transfected is incubated for the same time without the test compound to provide a negative control.

RNA is isolated from the two cultures as described in Chirgwin et al., Biochem. 18, 5294-99, 1979). Northern blots are prepared using 20 to 30 μg total RNA and hybridized with a ³²P-labeled ADAM-TS-like protein-specific probe at 65° C. in Express-hyb (CLONTECH). The probe comprises at least 11 contiguous nucleotides selected from the complement of SEQ ID NO: 1. A test compound which decreases the ADAM-TS-like protein-specific signal relative to the signal obtained in the absence of the test compound is identified as an inhibitor of ADAM-TS-like gene expression.

### EXAMPLE 5

Tissue-Specific Expression of ADAM-TS-Like Gene.

As a first step to establishing a role for ADAM-TS-like protein in the pathogenesis of COPD, expression profiling of the gene was done using real-time quantitative PCR (TaqMan) with RNA samples isolated from a wide range of human cells and tissues. Total RNA samples were either purchased from commercial suppliers or purified in-house. Two panels of RNAs were used for profiling: a whole body organ panel (Table 1) and a respiratory specific panel (Table 2).

Real-time quantitative PCR. This technique is a development of the kinetic analysis of PCR first described by Higuchi et al. (BioTechnology 10, 413-17, 1992; BioTechnology 11, 1026-30, 1993). The principle is that at any given cycle within the exponential phase of PCR, the amount of product is proportional to the initial number of template copies. PCR amplification is performed in the presence of an oligonucleotide probe (TaqMan probe) that is complementary to the target sequence and labeled with a fluorescent reporter dye and a quencher dye. During the extension phase of PCR, the probe is cleaved by the 5′-3′ endonuclease activity of Taq DNA polymerase, releasing the fluorophore from the effect of the quenching dye (Holland et al., Proc. Natl. Acad. Sci. U.S.A. 88, 7276-80, 1991). Because the fluorescence emission increases in direct proportion to the amount of the specific amplified product, the exponential growth phase of PCR product can be detected and used to determine the initial template concentration (Heid et al., Genome Res. 6, 986-94, 1996, and Gibson et al., Genome Res. 6, 995-1001, 1996).

RNA extraction and cDNA preparation. Total RNA from each of the ‘in-house’ samples listed in Table 2 was isolated using Qiagen's (Crawley, West Sussex, UK) RNeasy system according to the manufacturer's protocol. The concentration of purified RNA was determined using RiboGreen RNA quantitation kit (Molecular Probes Europe, The Netherlands). RNA concentrations of the samples purchased from commercial suppliers were also determined using RiboGreen. For the preparation of cDNA, 1 μg of total RNA was reverse transcribed using 200U of SUPERSCRIPT™ II RNaseH⁻ Reverse Transcriptase (Life Technologies, Paisley, UK), 10 mM dithiothreitol, 0.5 mM of each dNTP, and 5 μM random hexamers (PE Applied Biosystems, Warrington, Cheshire, UK) in a final volume of 20 μl according to the manufacturer's protocol.

TaqMan quantitative analysis. Specific primers and probe were designed according to the recommendations of PE Applied Biosystems and are listed below:

Forward primer:
5′-TCCCTGAATCAGGACTTCCCT-3′
Reverse primer:
5′-GTGCCGCAAGCCTGTGTT-3′
Probe:
5′-(FAM)-TGTTGGGCCTGAGAAACCGCACC-3′

where FAM=6-carboxy-fluorescein.

Quantitative PCR was performed with long of reverse transcribed RNA from each sample. Each determination was done in duplicate.

The assay reaction mix was as follows: IX final TaqMan Universal PCR Master Mix (from 2× stock) (PE Applied Biosystems, CA); 900 nM forward primer; 900 nM reverse primer; 200 nM probe; 10 ng cDNA; and water to 25 μl.

Each of the following steps were carried out once: pre PCR, 2 minutes at 50° C., and 10 minutes at 95° C. The following steps are carried out 40 times: denaturation, 15 seconds at 95° C., annealing/extension, 1 minute at 60° C.

Real-time quantitative PCR was done using an ABI Prism 7700 Sequence Detector. The C_{T} value generated for each reaction was used to determine the initial template concentration (copy number) by interpolation from a universal standard curve. The level of expression of the target gene in each sample was calculated relative to the sample with the lowest expression of the gene.

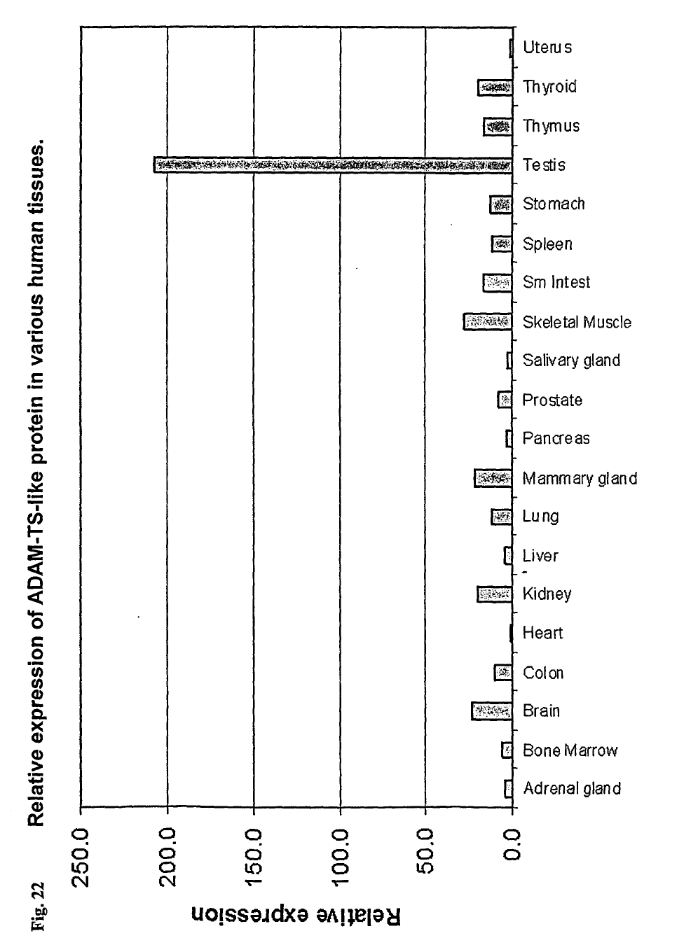

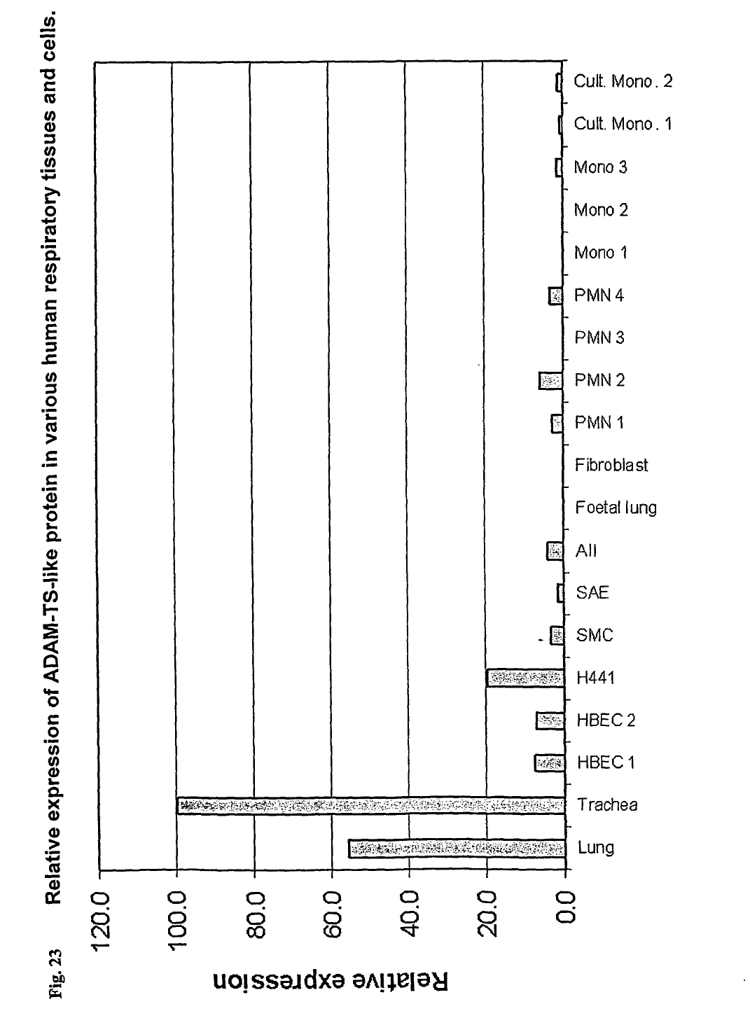

The relative expression of ADAM-TS-like gene across various human tissues is shown in FIG. 22. By far the highest level of expression was in testis, although mRNA transcripts were detected in all of the tissues tested. Of particular interest was the expression of ADAM-TS-like gene in lung and this was investigated further by analyzing the expression of the gene in some of the constituent cell types of the lung. In these samples, the level of expression was low in whole lung and trachea and very low or not expressed in all of the other cells (FIG. 23).

Although the function of ADAM-TS-like protein in lung is not known, it may be involved in the processing of peptides or proteins at cell surfaces, particularly in the upper airways. Dysfunction or dysregulation of the protease could play an important role in the pathogenesis of COPD. ADAM-TS-like protein, therefore, represents a potential therapeutic target for COPD.

TABLE 1
Human organ RNA panel used for real-time quantitative PCR.
All samples were obtained from
Clontech UK Ltd, Basingstoke, UK.

| Tissue | Cat. # |
|---|---|
| Adrenal gland | Human Panel V, K4004-1 |
| Bone marrow | Human Panel II, K4001-1 |
| Brain | Human Panel I, K4000-1 |
| Colon | Human Panel II, K4001-1 |
| Heart | Human Panel III, K4002-1 |
| Kidney | Human Panel I, K4000-1 |
| Liver | Human Panel I, K4000-1 |
| Lung | Human Panel I, K4000-1 |
| Mammary gland | Human Panel III, K4002-1 |
| Pancreas | Human Panel V, K4004-1 |
| Prostate | Human Panel III, K4002-1 |
| Salivary gland | Human Panel V, K4004-1 |
| Skeletal muscle | Human Panel III, K4002-1 |
| Small intestine | Human Panel II, K4001-1 |
| Spleen | Human Panel II, K4001-1 |
| Stomach | Human Panel II, K4001-1 |
| Testis | Human Panel III, K4002-1 |
| Thymus | Human Panel II, K4001-1 |
| Thyroid | Human Panel V, K4004-1 |
| Uterus | Human Panel III, K4002-1 |

TABLE 2
Human respiratory specific RNA panel
used for real-time quantitative PCR.

| Tissue/cell type | Supplier, cat # |
|---|---|
| Lung (fetal) | Takara (Japan) |
| Lung | Clontech, Human Panel I, K4000-1 |
| Trachea | Clontech, Human Panel I, K4000-1 |
| Cultured human bronchial epithelial | In-house |
| cells | |
| Cultured airway smooth muscle cells | In-house |
| Cultured small airway epithelial cells | In-house |
| Primary cultured alveolar type II cells | In-house |
| Cultured H441 cells (Clara-like) | In-house |
| Cultured lung fibroblasts | BioWhittaker, UK, CC2512 |
| Freshly isolated polymorphonuclear | In-house |
| leukocytes (neutrophils) | |
| Freshly isolated monocytes | In-house |
| Cultured monocytes (macrophage- | In-house |
| like) | |

1. An isolated polynucleotide encoding a ADAM-TS-like protein polypeptide and being selected from the group consisting of:
a) a polynucleotide encoding a ADAM-TS-like protein polypeptide comprising an amino acid sequence selected form the group consisting of:
amino acid sequences which are at least about 50% identical to the amino acid sequence shown in SEQ ID NO: 2; and
the amino acid sequence shown in SEQ ID NO: 2.
b) a polynucleotide comprising the sequence of SEQ ID NO: 1;
c) a polynucleotide which hybridizes under stringent conditions to a polynucleotide specified in (a) and (b);
d) a polynucleotide the sequence of which deviates from the polynucleotide sequences specified in (a) to (c) due to the degeneration of the genetic code; and
e) a polynucleotide which represents a fragment, derivative or allelic variation of a polynucleotide sequence specified in (a to (d).

2. An expression vector containing any polynucleotide of claim 1.

3. A host cell containing the expression vector of claim 2.

4. A substantially purified ADAM-TS-like protein polypeptide encoded by a polynucleotide of claim 1.

5. A method for producing a ADAM-TS-like protein polypeptide, wherein the method comprises the following steps:
a) culturing the host cell of claim 3 under conditions suitable for the expression of the ADAM-TS-like protein polypeptide; and
b) recovering the ADAM-TS-like protein polypeptide from the host cell culture.

6. A method for detection of a polynucleotide encoding a ADAM-TS-like protein polypeptide in a biological sample comprising the following steps:
a) hybridizing any polynucleotide of claim 1 to a nucleic acid material of a biological sample, thereby forming a hybridization complex; and
b) detecting said hybridization complex.

7. The method of claim 6, wherein before hybridization, the nucleic acid material of the biological sample is amplified.

8. A method for the detection of a polynucleotide of claim 1 or a ADAM-TS-like protein polypeptide of claim 4 comprising the steps of:
contacting a biological sample with a reagent which specifically interacts with the polynucleotide or the ADAM-TS-like protein polypeptide.

9. A diagnostic kit for conducting the method of any one of claims 6 to 8.

10. A method of screening for agents which decrease the activity of a ADAM-TS-like protein, comprising the steps of:
contacting a test compound with any ADAM-TS-like protein polypeptide encoded by any polynucleotide of claim 1;
detecting binding of the test compound to the ADAM-TS-like protein polypeptide, wherein a test compound which binds to the polypeptide is identified as a potential therapeutic agent for decreasing the activity of a ADAM-TS-like protein.

11. A method of screening for agents which regulate the activity of a ADAM-TS-like protein, comprising the steps of:
contacting a test compound with a ADAM-TS-like protein polypeptide encoded by any polynucleotide of claim 1; and
detecting a ADAM-TS-like protein activity of the polypeptide, wherein a test compound which increases the ADAM-TS-like protein activity is identified as a potential therapeutic agent for increasing the activity of the ADAM-TS-like protein, and wherein a test compound which decreases the ADAM-TS-like protein activity of the polypeptide is identified as a potential therapeutic agent for decreasing the activity of the ADAM-TS-like protein.

12. A method of screening for agents which decrease the activity of a ADAM-TS-like protein, comprising the steps of:
contacting a test compound with any polynucleotide of claim 1 and detecting binding of the test compound to the polynucleotide, wherein a test compound which binds to the polynucleotide is identified as a potential therapeutic agent for decreasing the activity of ADAM-TS-like protein.

13. A method of reducing the activity of ADAM-TS-like protein, comprising the steps of:
contacting a cell with a reagent which specifically binds to any polynucleotide of claim 1 or any ADAM-TS-like protein polypeptide of claim 4, whereby the activity of ADAM-TS-like protein is reduced.

14. A reagent that modulates the activity of a ADAM-TS-like protein polypeptide or a polynucleotide wherein said reagent is identified by the method of any of the claim 10 to 12.

15. A pharmaceutical composition, comprising:
the expression vector of claim 2 or the reagent of claim 14 and a pharmaceutically acceptable carrier.

16. Use of the pharmaceutical composition of claim 15 for modulating the activity of a ADAM-TS-like protein in a disease.

17. Use of claim 16 wherein the disease is a cardiovascular or a liver disorder or COPD.

18. A cDNA encoding a polypeptide comprising the amino acid sequence shown in SEQ ID NO: 2.

19. The cDNA of claim 18 which comprises SEQ ID NO: 1.

20. The cDNA of claim 18 which consists of SEQ ID NO: 1.

21. An expression vector comprising a polynucleotide which encodes a polypeptide comprising the amino acid sequence shown in SEQ ID NO: 2.

22. The expression vector of claim 21 wherein the polynucleotide consists of SEQ ID NO: 1.

23. A host cell comprising an expression vector which encodes a polypeptide comprising the amino acid sequence shown in SEQ ID NO: 2.

24. The host cell of claim 23 wherein the polynucleotide consists of SEQ ID NO: 1.

25. A purified polypeptide comprising the amino acid sequence shown in SEQ ID NO: 2.

26. The purified polypeptide of claim 25 which consists of the amino acid sequence shown in SEQ ID NO: 2.

27. A fusion protein comprising a polypeptide having the amino acid sequence shown in SEQ ID NO: 2.

28. A method of producing a polypeptide comprising the amino acid sequence shown in SEQ ID NO: 2, comprising the steps of:
culturing a host cell comprising an expression vector which encodes the polypeptide under conditions whereby the polypeptide is expressed; and
isolating the polypeptide.

29. The method of claim 28 wherein the expression vector comprises SEQ ID NO: 1.

30. A method of detecting a coding sequence for a polypeptide comprising the amino acid sequence shown in SEQ ID NO: 2, comprising the steps of:
hybridizing a polynucleotide comprising 11 contiguous nucleotides of SEQ ID NO: 1 to nucleic acid material of a biological sample, thereby forming a hybridization complex; and
detecting the hybridization complex.

31. The method of claim 30 further comprising the step of amplifying the nucleic acid material before the step of hybridizing.

32. A kit for detecting a coding sequence for a polypeptide comprising the amino acid sequence shown in SEQ ID NO: 2, comprising:
a polynucleotide comprising 11 contiguous nucleotides of SEQ ID NO: 1; and
instructions for the method of claim 30.

33. A method of detecting a polypeptide comprising the amino acid sequence shown in SEQ ID NO: 2, comprising the steps of:
contacting a biological sample with a reagent that specifically binds to the polypeptide to form a reagent-polypeptide complex; and
detecting the reagent-polypeptide complex.

34. The method of claim 33 wherein the reagent is an antibody.

35. A kit for detecting a polypeptide comprising the amino acid sequence shown in SEQ ID NO: 2, comprising:
an antibody which specifically binds to the polypeptide; and
instructions for the method of claim 33.

36. A method of screening for agents which can modulate the activity of a human ADAM-TS-like protein, comprising the steps of:
contacting a test compound with a polypeptide comprising an amino acid sequence selected from the group consisting of: (1) amino acid sequences which are at least about 50% identical to the amino acid sequence shown in SEQ ID NO: 2 and (2) the amino acid sequence shown in SEQ ID NO: 2; and
detecting binding of the test compound to the polypeptide, wherein a test compound which binds to the polypeptide is identified as a potential agent for regulating activity of the human ADAM-TS-like protein.

37. The method of claim 36 wherein the step of contacting is in a cell.

38. The method of claim 36 wherein the cell is in vitro.

39. The method of claim 36 wherein the step of contacting is in a cell-free system.

40. The method of claim 36 wherein the polypeptide comprises a detectable label.

41. The method of claim 36 wherein the test compound comprises a detectable label.

42. The method of claim 36 wherein the test compound displaces a labeled ligand which is bound to the polypeptide.

43. The method of claim 36 wherein the polypeptide is bound to a solid support.

44. The method of claim 36 wherein the test compound is bound to a solid support.

45. A method of screening for agents which modulate an activity of a human ADAM-TS-like protein, comprising the steps of:
contacting a test compound with a polypeptide comprising an amino acid sequence selected from the group consisting of: (1) amino acid sequences which are at least about 50% identical to the amino acid sequence shown in SEQ ID NO: 2 and (2) the amino acid sequence shown in SEQ ID NO: 2; and
detecting an activity of the polypeptide, wherein a test compound which increases the activity of the polypeptide is identified as a potential agent for increasing the activity of the human ADAM-TS-like protein, and wherein a test compound which decreases the activity of the polypeptide is identified as a potential agent for decreasing the activity of the human ADAM-TS-like protein.

46. The method of claim 45 wherein the step of contacting-is in a cell.

47. The method of claim 45 wherein the cell is in vitro.

48. The method of claim 45 wherein the step of contacting is in a cell-free system.

49. A method of screening for agents which modulate an activity of a human ADAM-TS-like protein, comprising the steps of:
contacting a test compound with a product encoded by a polynucleotide which comprises the nucleotide sequence shown in SEQ ID NO: 1; and
detecting binding of the test compound to the product, wherein a test compound which binds to the product is identified as a potential agent for regulating the activity of the human ADAM-TS-like protein.

50. The method of claim 49 wherein the product is a polypeptide.

51. The method of claim 49 wherein the product is RNA.

52. A method of reducing activity of a human ADAM-TS-like protein, comprising the step of:
contacting a cell with a reagent which specifically binds to a product encoded by a polynucleotide comprising the nucleotide sequence shown in SEQ ID NO: 1, whereby the activity of a human ADAM-TS-like protein is reduced.

53. The method of claim 52 wherein the product is a polypeptide.

54. The method of claim 53 wherein the reagent is an antibody.

55. The method of claim 52 wherein the product is RNA.

56. The method of claim 55 wherein the reagent is an antisense oligonucleotide.

57. The method of claim 56 wherein the reagent is a ribozyme.

58. The method of claim 52 wherein the cell is in vitro.

59. The method of claim 52 wherein the cell is in vivo.

60. A pharmaceutical composition, comprising:
a reagent which specifically binds to a polypeptide comprising the amino acid sequence shown in SEQ ID NO: 2; and
a pharmaceutically acceptable carrier.

61. The pharmaceutical composition of claim 60 wherein the reagent is an antibody.

62. A pharmaceutical composition, comprising:
a reagent which specifically binds to a product of a polynucleotide comprising the nucleotide sequence shown in SEQ ID NO: 1; and
a pharmaceutically acceptable carrier.

63. The pharmaceutical composition of claim 62 wherein the reagent is a ribozyme.

64. The pharmaceutical composition of claim 62 wherein the reagent is an antisense oligonucleotide.

65. The pharmaceutical composition of claim 62 wherein the reagent is an antibody.

66. A pharmaceutical composition, comprising:
an expression vector encoding a polypeptide comprising the amino acid sequence shown in SEQ ID NO: 2; and
a pharmaceutically acceptable carrier.

67. The pharmaceutical composition of claim 66 wherein the expression vector comprises SEQ ID NO: 1.

68. A method of treating a ADAM-TS-like protein dysfunction related disease, wherein the disease is selected from a cardiovascular or a liver disorder or COPD, comprising the step of:
administering to a patient in need thereof a therapeutically effective dose of a reagent that modulates a function of a human ADAM-TS-like protein, whereby symptoms of the ADAM-TS-like protein dysfunction related disease are ameliorated.

69. The method of claim 68 wherein the reagent is identified by the method of claim 36.

70. The method of claim 68 wherein the reagent is identified by the method of claim 45.

71. The method of claim 68 wherein the reagent is identified by the method of claim 49.
